(19) United States
(12) Patent Application Publication
(10) Pub. No.: US 2024/0335412 A1
(43) Pub. Date: Oct. 10, 2024

(54) WEARABLE PET DEVICES WITH PASSIVE DIFFUSION OF SEMIOCHEMICAL COMPOSITIONS

(71) Applicant: VIRBAC, Carros (FR)

(72) Inventors: Aurélie GIRARDIN, Saint Laurent du Var (FR); Patricia MONGINOUX, Villeneuve Loubet (FR); Cécile LE JEANNE, Lussac les Eglises (FR); Jean JOLIVET, Chaillac (FR)

(21) Appl. No.: 18/257,569

(22) PCT Filed: Dec. 17, 2021
(86) PCT No.: PCT/EP2021/086599

§ 371 Date: Jun. 14, 2023

(30) Foreign Application Priority Data
Dec. 21, 2020 (FR) FR2013842

(51) Int. Cl. A61K 31/231; A61K 9/00; A61K 31/23; A61K 47/02; A61K 47/32; A61P 25/22

(52) U.S. Cl. CPC A61K 31/231; A61K 9/007; A61K 31/23; A61K 47/02; A61K 47/32; A61P 25/22

(57) ABSTRACT

The present invention relates to portable pet devices capable of passively diffusing semiochemical compositions; these soothing devices allow animals to better manage stress-generating situations (situations of separation, withdrawal, transport, fear of noises, fireworks, etc.) and allow them to better adapt to the new situations.

### CROSS-REFERENCE TO RELATED APPLICATIONS

This application is a National Phase Patent Application and claims priority to and the benefit of International Patent Application No. PCT/EP2021/086599, filed on Dec. 17, 2021, which claims priority to and the benefit of French Patent Application No. FR2013842, filed on Dec. 21, 2020. The entire contents of both of which are incorporated herein by reference.

The present invention relates to wearable pet devices capable of passively diffusing semiochemical compositions.

The present invention relates in particular to devices capable of diffusing semiochemical compositions for a longer period of time than the devices known in the prior art.

Devices capable of passively diffusing semiochemical compositions are known in the prior art. The semiochemical compositions are compounds or mixtures of compounds naturally secreted by the animals to communicate within a same species. For example, during the lactation the female dog secretes a pheromone to reassure her puppies when they are near her. Similarly, cats secrete facial pheromones on their face and deposit them in places known to mark a familiar territory.

Numerous studies have been carried out to identify these mixtures of compounds and reproduce them synthetically.

Such mixtures of compounds are, for example, indicated to help dogs to better manage the stressful situations (separation situations, weaning, transport, fear of noise, fireworks etc.) and to allow them to better adapt to new situations.

The products concerned are compounds of, in particular, sprays, active diffusers (electric plugs heating a semiochemical composition dispersed or solubilised in an organic solvent) and collars.

The prior art collars, in particular those of the ADAPTIL® brand, whose active principle or semiochemical active is the synthetic analogue of the pheromone secreted by the female dog to reassure her puppies on contact with her, contain a polymer, ethylene-vinyl acetate or EVA, and a semiochemical composition consisting of a mixture of fat acid methyl esters. The semiochemical composition, impregnated in the polymer, diffuses passively into the air where it is perceived by the vomeronasal organ of the dog.

The diffusion rate of this collar (or release rate) is such that the collar can be used for four weeks according to the manufacturer. The applicant was able to demonstrate that after four weeks, only a minor fraction of the semiochemical composition had actually diffused out of the device. Thus, after four weeks, a maximum of 14% of the semiochemical composition initially present in the collar has diffused.

These results clearly indicate that the impregnation of the semiochemical composition in ethylene vinyl acetate does not allow the complete release of the semiochemical composition, and that after four weeks, the release kinetics of the pheromone become very low, which no longer allows the device to be effective.

It is therefore an object of the present invention to improve the devices of the prior art, in particular the pet collars, containing a semiochemical composition.

A first objective of the present invention is to allow a diffusion for a longer period, in particular more than one month, of the semiochemical composition compared to the devices of the prior art.

A second objective of the present invention is to allow the diffusion of a higher relative amount, with respect to the total amount in the device, of the semiochemical composition out of the device compared to the devices of the prior art.

A third objective of the present invention is to allow a controlled diffusion, i.e. the release of an equivalent average amount of semiochemical composition over successively at least two equal time intervals per time period compared to the devices of the prior art.

A fourth objective of the present invention is to provide a device preferably fulfilling the first objective and the second objective which retains a sufficient flexibility to be transformed into all kinds of objects usable on animals, in particular on pets, in particular collars.

A fifth objective of the present invention is to obtain a device allowing a better compliance of the treatment by the owner, in particular by increasing the duration of action of the device and by increasing the time between two applications of the device on the animal or in the immediate environment of the animal.

Unless otherwise stated, all percentages are given as the weight of a component in relation to the total weight of the components shown (m/m).

A first object of the present invention is a device, in particular for continuously releasing onto an animal or into the immediate environment of an animal a semiochemical composition for pets, comprising:
a polymer matrix,
a semiochemical composition,
a control agent controlling the diffusion of the semiochemical composition out of the device,
possibly an antioxidant,
possibly an anti-UV agent,
possibly a flow agent.

According to this first object, the present invention relates to a device for continuously releasing onto an animal or into the immediate environment of an animal a semiochemical composition for pets comprising:
a polymer matrix,
a semiochemical composition comprising a mixture of volatile fat acid derivatives,
a control agent controlling the diffusion of the semiochemical composition out of the device selected from a clay, a diatomite and a zeolite.

The devices according to the present invention include in particular solid shaped bodies that can be worn by the animal or installed in the immediate environment of the animal, such as collars, harnesses, collar pendants (medallions), ear tags, collars that can be attached to limbs or parts of the body, adhesive strips and films. Preference is given to the medallions and the collars, especially to the collars.

The immediate environment of the animal is defined as the areas where it is likely to evolve, such as the household of its owner, its doghouse, a vehicle, a transport cage, etc.

The shaped bodies according to the invention can also take the form of balls that the user can place in a container.

The devices can also take the form of objects in the environment of the animal such as bowls, floor mats, blankets, clothing or training mats.

The size and the shape of any device is adapted to the animal for which it is intended; the device is equipped, if necessary, with an attachment means known to the person skilled in the art. For example, for the systems for closing the collars consisting of an extruded strip, the attachment may be plastic moulded with its rivet, attached with a metal rivet, attached with a metal clip, or it may be a metal loop system with loops held in the folded strap by several rivets.

In the case of collars, it is also possible to offer collars of a standard length that will be cut by the owner of the pet to best fit the size of the neck of the target animal, for example, a puppy, a small dog, a medium dog or a large dog.

In one embodiment, the device according to the invention is a collar of a predetermined length to which a loop is attached allowing to hold the excess length of the collar against the part of the collar in contact with the neck of the animal. The loop allows that the entire length of the collar in which the semiochemical composition is present is retained and that this excess length does not bother the animal or the owner.

The device may have a smooth surface or it may have reliefs that increase the exchange surface and the amount of semiochemical actives released in a given time.

In the context of the present invention, the device is therefore a composite comprising a polymer matrix as a continuous phase in which is dispersed a control agent controlling the diffusion of the semiochemical composition out of the device. The terms “composites” and “polymer matrix” are understood in the sense of the present invention in their common meaning for the person skilled in the art (e.g. as defined in “Compendium-of-Polymer-Terminology-and-Nomenclature-IUPAC-Recommendations-2008”). The term “polymer matrix” refers to a matrix comprising a polymer with a continuous, non-foamed or honeycomb structure and therefore excludes the polymeric foams and the polymeric materials with a honeycomb or cellular structure.

The polymer matrix must have a sufficient strength and flexibility to ensure that the device does not break or become brittle during its manufacture and its use. The polymer matrix must also be durable enough to withstand the normal wear and tear when the device is worn by, or applied to, the animal. Furthermore, the polymer matrix must allow an adequate migration, i.e. the passive diffusion of the semiochemical composition to the surface of the device as well as the release of said composition onto the animal or into the environment of the animal, in particular its olfactory apparatus.

Thermoplastic polymers, elastomers and thermoplastic polyvinyl resins are advantageously used. Thermoplastic and flexible polyolefins and elastomers are suitable for a use as a carrier substance or base for the shaped bodies. These include polyvinyl resins, EPDM (ethylene/propylene-diene terpolymer), polyethylene (e.g. HDPE or LLDPE) and polypropylene, which are sufficiently compatible with the above-mentioned active compounds.

The polyvinyl resins comprise polyvinyl halides, such as polyvinyl chloride, vinyl polyvinyl acetate chloride and polyvinyl fluoride; polyvinylbenzenes, such as polystyrene and polyvinyltoluene; and vinyl acetate copolymers, such as thermoplastic copolymer of ethylene-vinyl acetate or EVA.

Other plastic materials suitable for use as a matrix for the devices according to the invention are thermoplastic elastomers. Examples include thermoplastic polyolefins (TPO).

Advantageously, the above-mentioned polyvinyl resins are used. Preferably, the thermoplastic copolymers of ethylene-vinyl acetate (EVA) are used; the properties, in particular flexibility and strength, of these copolymers vary according to their vinyl acetate content (also referred to as grade), preferably thermoplastic copolymers of ethylene-vinyl acetate having a vinyl acetate content of between 5 and 20%, preferably between 5 and 16%, and even more preferably around 9%. These grades of EVA are particularly useful for the manufacture of animal collars.

The semiochemical composition used is preferably a soothing semiochemical composition comprising a mixture of volatile fat acid derivatives.

For the purposes of the present invention, a soothing semiochemical composition qualifies a mixture of chemical substances having signal value between living beings of natural origin, for example isolated from a living being, or of synthetic origin, for example obtained by mixing volatile derivatives of a fat acid capable of producing a relaxing or soothing effect on the animal.

For the purposes of this invention, “volatile fat acid derivative” means any fat acid derivative capable of being diffused onto the animal or into the environment of the animal without the application of an external electrical heat source to the device.

For the purposes of this invention, “fat acid” means a saturated or unsaturated, linear or branched hydrocarbon monocarboxylic or dicarboxylic acid having between 4 and 22 carbon atoms. Advantageously, the fat acid is selected from the group consisting of oleic acid, palmitic acid, azelaic acid, pimelic acid, capric acid, lauric acid, myristic acid, palmitoleic acid, linoleic acid, stearic acid, arachidonic acid, n-butyric acid, isobutyric acid, α-methylbutyric acid, caproic acid, pivalic acid, γ-linoleic acid, eicosapentanoic acid, pentadecanoic acid, tridecanoic acid, or docosahexanoic acid.

The volatile fat acid derivatives include fat acid esters, such as esters formed from the fat acid and an alcohol in C₁ to C₆. Advantageously, the fat acid ester is ethyl or methyl ester. Preferably, the fat acid ester is the fat acid methyl ester.

Advantageously, the soothing semiochemical composition thus comprises a mixture of volatile fat acid derivatives wherein the fat acid is selected from the group consisting of oleic acid, palmitic acid, azelaic acid, pimelic acid, capric acid, lauric acid, myristic acid, palmitoleic acid, linoleic acid, stearic acid, arachidonic acid, n-butyric acid, isobutyric acid, α-methylbutyric acid, caproic acid, pivalic acid, γ-linoleic acid, eicosapentanoic acid, pentadecanoic acid, tridecanoic acid, docosahexanoic acid, advantageously selected from the group consisting of oleic acid, palmitic acid, lauric acid, myristic acid, linoleic acid, stearic acid and pentadecanoic acid.

More advantageously, the soothing semiochemical composition comprises a mixture of volatile fat acid derivatives selected from the group consisting of methyl oleate, methyl palmitate, methyl azelate, methyl pimelate, methyl caprate, methyl laurate, methyl myristate, methyl palmitoleate, methyl linoleate, methyl stearate, methyl arachidonate, methyl n-butyrate, methyl isobutyrate, methyl α-methylbutyrate, methyl caproate, methyl pivalate, methyl γ-linoleate, methyl eicosapentanoate, methyl pentadecanoate, methyl tridecanoate, methyl docosahexanoate, advantageously selected from the group consisting of methyl oleate, methyl palmitate, methyl laurate, methyl myristate, methyl linoleate, methyl stearate and methyl pentadecanoate.

The volatile fat acid derivatives and their relative proportions in the soothing semiochemical composition depend on the target animal.

When the target animal is a dog, the soothing semiochemical composition may be selected from those described in international application WO2009144321.

Particular mixtures of esters, preferably methyl esters, of fat acids for soothing the dogs are esters of the following fat acids:
a mixture A of oleic acid and n-butyric acid;
a mixture B of oleic acid, palmitic acid and linoleic acid;
a mixture C of oleic acid, palmitic acid, linoleic acid and palmitoleic acid;
a mixture D of capric acid, lauric acid, myristic acid, palmitoleic acid, palmitic acid, linoleic acid and oleic acid;
a mixture E of oleic acid, palmitic acid, linoleic acid and myristic acid;
a mixture F of oleic acid, palmitic acid, linoleic acid, lauric acid and myristic acid;
a mixture G of oleic acid, palmitic acid, linoleic acid, myristic acid and pentadecanoic acid;
a mixture H of oleic acid, palmitic acid, linoleic acid, myristic acid, pentadecanoic acid and stearic acid;
a mixture I of oleic acid, palmitic acid, linoleic acid, myristic acid, lauric acid and pentadecanoic acid;
a mixture J of lauric acid, myristic acid, pentadecanoic acid, palmitic acid, stearic acid, oleic acid and linoleic acid;
a mixture K of oleic acid, azelaic acid, pimelic acid and palmitic acid.

Mixtures of esters, preferably methyl esters, of fat acids are esters of the following fat acids:
mixture L comprising about 55-65% oleic acid, and 45-35% palmitic acid;
mixture M comprising about 45% oleic acid, 16% azelaic acid, 18% pimelic acid, and 21% palmitic acid;
mixture N comprising about 30% palmitic acid, 30% oleic acid, and 40% linoleic acid;
mixture O comprising about 30% palmitic acid, 40% linoleic acid, 10% palmitoleic acid, and 20% oleic acid.

A particular fat acid ester mixture P for soothing the dogs comprises about 35% methyl oleate, about 2% methyl laurate, about 13% methyl stearate, about 21% methyl linoleate, about 5% methyl myristate, about 4% methyl pentadecanoate, and about 20% methyl palmitate.

The inventors of the present invention have furthermore demonstrated that a soothing semiochemical composition not comprising methyl pentadecanoate leads to a similar effect on the dog as the mixture P. As pentadecanoic acid is a relatively rare fat acid in nature, whose main origin is the fat fraction of cow's milk where it is present in small quantities (1.2%), it is difficult to obtain at a reasonable cost. The discovery that the absence of this fat acid does not reduce the soothing effectiveness of a semiochemical composition therefore advantageously reduces the costs associated with the manufacture of the devices according to the invention. A further particular mixture Q of fat acid esters for soothing the dogs thus comprises between 32% and 38% methyl oleate, between 1% and 3% methyl laurate, between 13% and 16% methyl stearate, between 18% and 24% methyl linoleate, between 3% and 7% methyl myristate and between 18% and 24% methyl palmitate.

A more particular mixture R of fat acid esters for soothing the dogs comprises about 35% methyl oleate, about 2% methyl laurate, about 15.5% methyl stearate, about 21% methyl linoleate, about 5% methyl myristate and about 21% methyl palmitate.

When the target animal is a cat, the soothing semiochemical composition may be selected from those described in the international application WO2015140631.

The soothing semiochemical composition is, for example, a soothing cat pheromone that is derived from skin secretions from the area between the shoulder blades and the ears of cats.

The semiochemical composition for soothing the cats therefore advantageously comprises methyl palmitate, methyl linoleate, methyl oleate, methyl stearate, methyl laurate and methyl myristate.

A particular mixture S of fat acid ester for soothing the cats comprises between 15% and 25% methyl palmitate, between 10% and 27% methyl linoleate, between 25% and 35% methyl oleate, between 5% and 15% methyl stearate, between 5% and 15% methyl laurate and between 5% and 15% methyl myristate.

Another particular mixture T of fat acid esters for soothing the cats comprises between 19% and 26% methyl palmitate, between 14% and 20% methyl linoleate, between 26% and 32% methyl oleate, between 8% and 14% methyl stearate, between 8% and 14% methyl laurate and between 8% and 14% methyl myristate.

An even more particular mixture U comprises 22% methyl palmitate, 17% methyl linoleate, 28% methyl oleate, 11% methyl stearate, 11% methyl laurate and 11% methyl myristate.

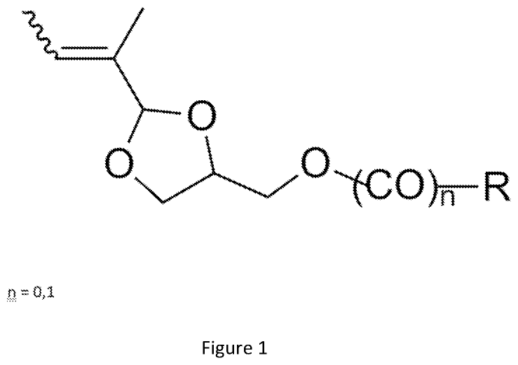

Another semiochemical composition that can be used in the devices according to the invention is described in the patent U.S. Pat. No. 10,227,321, wherein at least one volatile fat acid derivative is an ester of the formula according to FIG. 1, wherein n=0 and R is a saturated or unsaturated linear alkyl comprising from 9 to 30 carbon atoms or n=1 and R is a saturated or unsaturated linear alkyl comprising from 8 to 22 carbon atoms.

The target animal is a non-human mammal. These may be pets such as cats, dogs, ferrets and rabbits, or livestock for which it is useful to improve the living conditions such as rabbits and pigs. The person skilled in the art will use in the device according to the invention the semiochemical composition capable of preventing the manifestations of stress and/or reducing the anxiety adapted to each target species.

The amount of semiochemical composition in the device can vary widely. The proportion of semiochemical composition is typically between 0.1% and 25% by weight of the total weight of the device. Advantageously, in a collar the semiochemical composition represents from 1 to 10% by weight, more advantageously from 3 to 8% by weight, preferably about 5% of the total weight of the collar.

For the purposes of this invention, “approximately x %” means a variation of plus or minus 0.2% from the stated percentage.

The devices according to the present invention differ from those described in the prior art, in particular from the collar marketed under the trademark ADAPTIL®, in that they contain a control agent controlling the diffusion of the semiochemical composition out of the device capable of slowing down the diffusion of said semiochemical composition.

The applicant has shown that the introduction of materials capable of adsorbing the semiochemical composition allows to control the diffusion of said semiochemical composition. “Controlling the diffusion of the semiochemical composition” is understood as releasing a higher relative amount of semiochemical composition and/or for a longer period of time and/or releasing an equivalent average amount of semiochemical composition over successively at least two equal time intervals, e.g. releasing about 10% of the semiochemical composition over a period of 14 days for at least two periods, compared to the same device not containing the material capable of adsorbing the semiochemical composition; these properties result in a higher relative amount, with respect to the total amount in the device, of the semiochemical composition being diffused out of the device compared to the devices of the prior art.

Among the control agents controlling the diffusion of the semiochemical composition out of the device the include inorganic materials such as clays, diatomites and zeolites. Advantageously, clays with a chain structure (hornblende-like silica tetrahedron chains) are used, in particular phyllosilicates such as wollastonite (e.g. sold under the name casiflux F75), bentonite, kaolinite, montmorillonite, attapulgite, sepiolite and paligorskyte. Attapulgite, sepiolite and paligorskyte are preferred, with sepiolite being even more preferred.

Although sepiolite extracted from the ground is suitable, chemically treated (e.g. to change its zeta potential) or thermally treated (e.g. to change its structure) sepiolites can also be used.

The control agent controlling the diffusion of the semiochemical composition out of the device is introduced into the device in proportions varying from 5 to 25%, advantageously from 10 to 20%, preferably about 15% by weight in relation to the total weight of the device.

The devices according to the present invention may also comprise ingredients conventionally used to facilitate the shaping of the device, for example a flow agent, a plasticiser, an antioxidant, an anti-UV agent or any other ingredient allowing to improve the stability over time.

The role of the flow agent is to facilitate the flow of the mixture into the hopper that feeds the screw of the extruder. The flow agents are well known to the person skilled in the art. The flow agent is preferably selected from colloidal silica, magnesium stearate, even more preferably, the flow agent is colloidal silica.

The stability of the devices according to the present invention, and in particular the stability of the components of the semiochemical composition, can for example be improved by incorporating antioxidants or anti-UV agents.

Antioxidants include phenolic compounds such as butylated hydroxyanisole (BHA), butylated hydroxytoluene (BHT), gallic acid, propyl gallate and pyrogallol, hydroquinones such as tert-butylhydroquinone (TBHQ) and di-tert-butylhydroquinone (DTBHQ), tocopherols, amines such as octyl butyl diphenylamine (OBPA), poly (1,2-dihydro-2,2,4-trimethylquinoline (Orox PK), N,N′-phenyl-p-phenylenediamine (DPD) and ethoxyquinine, and a mixture of these antioxidants. Such compounds are well known to the person skilled in the art and can be chosen without undue effort; preferably, the antioxidant used is TBHQ. As an indication, the amount of antioxidants used can be between 0.001 and 0.1%.

The anti-UV agents allow to opacify the material of the device or to absorb the UV rays, thus preventing the light rays from reaching the core of the device and limiting the degradation of its components by light, in particular of the semiochemical composition. They can be chosen from carbon black, aluminium hydroxide (Al(OH)3), calcium carbonate and hindered amine light stabilizers (HALS), marketed for example under the name Hostavin N30P®.

According to a particular embodiment, the device according to the invention further comprises a flow agent such as colloidal silica and an ingredient to allow improving the stability over time of the semiochemical composition such as an antioxidant or an anti-UV agent.

The proportion of each of the ingredients in the device is chosen so that the diffusion of the semiochemical composition is suitable for the desired purpose while maintaining the rheological properties of the device (e.g. flexibility or elasticity in the case of an animal collar).

A second object of the present invention therefore relates to a device comprising:
a polymer matrix,
from 0.5 to 10% of a semiochemical composition as defined above, preferably about 5%,
from 5 to 25% of a control agent controlling the diffusion of the semiochemical composition out of the device as defined above, preferably about 15%,
optionally from 0.001 to 0.1% of an antioxidant, preferably about 0.005%,
optionally from 0.1 to 2% of an anti-UV agent, preferably about 1%,
optionally from 0.1 to 5% of a flow agent, preferably about 1.5%.

In a first embodiment, the device according to the present invention comprises:
a polymer matrix consisting of a thermoplastic copolymer of ethylene-vinyl acetate,
from 0.5 to 10% of a semiochemical composition as defined above, preferably about 5%,
from 5 to 25% of a control agent controlling the diffusion of the semiochemical composition out of the device as defined above, preferably about 15%,
optionally from 0.001 to 0.1% of an antioxidant, preferably about 0.05%,
optionally from 0.1 to 2% of an anti-UV agent, preferably about 1%,
optionally from 0.1 to 5% of a flow agent, preferably about 1.5%.

In a second embodiment, the device according to the present invention comprises:
a polymer matrix,
from 0.5 to 10% of a semiochemical composition, preferably about 5%, said semiochemical composition comprising a mixture of fat acid esters selected from laurate, myristate, palmitate, stearate, oleate and linoleate, and optionally pentadecanoate,
from 5 to 25% of a control agent controlling the diffusion of the semiochemical composition out of the device as defined above, preferably about 15%,
optionally from 0.001 to 0.1% of an antioxidant, preferably about 0.05%,
optionally from 0.1 to 2% of an anti-UV agent, preferably about 1%,
optionally from 0.1 to 5% of a flow agent, preferably about 1.5%.

In a third embodiment, the device according to the present invention comprises:
a polymer matrix,
from 0.5 to 10% of a semiochemical composition, preferably about 5%,
from 5 to 25% of a control agent controlling the diffusion of the semiochemical composition out of the device as defined above, preferably about 15%, said agent being a clay, in particular a phyllosilicate, in particular sepiolite, attapulgite or paligorskyte,
optionally from 0.001 to 0.1% of an antioxidant, preferably about 0.05%
optionally from 0.1 to 2% of an anti-UV agent, preferably about 1%,
optionally from 0.1 to 5% of a flow agent, preferably about 1.5%.

In a specific embodiment, the device according to the present invention comprises:
a polymer matrix consisting of a thermoplastic copolymer of ethylene-vinyl acetate,
from 0.5 to 10% of a semiochemical composition, preferably about 5%, said semiochemical composition comprising a mixture of fat acid esters selected from laurate, myristate, palmitate, stearate, oleate and linoleate, and optionally pentadecanoate,
from 5 to 25% of a clay, preferably selected from sepiolite, attapulgite and paligorskyte, preferably about 15%,
optionally from 0.001 to 0.1% of an antioxidant, preferably about 0.05%
optionally from 0.1 to 2% of an anti-UV agent, preferably about 1%,
optionally from 0.1 to 5% of a flow agent, preferably about 1.5%.

Preferably, the present invention relates to a dog collar, comprising:
a polymer matrix consisting of a thermoplastic copolymer of ethylene-vinyl acetate,
about 5% of a semiochemical composition, comprising a mixture of methyl laurate, methyl myristate, methyl palmitate, methyl stearate, methyl oleate and methyl linoleate, and optionally methyl pentadecanoate,
about 15% sepiolite,
about 0.05% of an antioxidant,
about 1% of a compound protecting from light,
about 1.5% of a flow agent.

In a third aspect, the present invention relates to a method for achieving one or more of the following effects:
ensuring the well-being of an animal in its environment,
helping an animal deal with stress and/or anxiety,
reducing the stress associated with separation in an animal,
reducing the stress associated with weaning an animal,
reducing the stress associated with loneliness in an animal,
reducing the stress in an animal before and/or during transport,
reducing the stress associated with noise such as thunderstorms or fireworks,
reducing the stress associated with a pathology, a medical treatment, a visit to a veterinarian that may require an examination or even a surgery,
reducing the aggressiveness of an animal,
improving the behaviour of an animal, especially an aggressive animal or a fearful animal,
producing a relaxing effect on the animal,
helping an animal to adapt to a new situation, for example when it is adopted, when a new animal or child arrives in its environment, when it moves house,
reducing the stress-related urine marking in an animal,
reducing the uncleanliness and the filth of an animal,
improving the conditions for familiarising an animal with its fellow creatures,
preventing the scratching or the destruction of the territory,
reducing the sound manifestations such as crying and barking,
generating a less aggressive, more relaxed and more affectionate behaviour, in particular with their masters, in an animal,

comprising the diffusion of a semiochemical composition from a device as described above, preferably for a period of more than 1 month, more advantageously more than 2 months, preferably for a period of about 3 months, the animal being a dog or a cat. Said diffusion takes place on the animal or in the immediate environment of the animal, i.e. close to its olfactory apparatus.

In a fourth aspect, the present invention relates to the use of a device as described above to achieve one or more of the following effects:
ensuring the well-being of an animal in its environment,
helping an animal deal with stress and/or anxiety,
reducing the stress associated with separation in an animal,
reducing the stress associated with weaning an animal,
reducing the stress associated with loneliness in an animal,
reducing the stress in an animal before and/or during transport,
reducing the stress associated with noise such as thunderstorms or fireworks,
reducing the stress associated with a pathology, a medical treatment, a visit to a veterinarian that may require an examination or even a surgery,
reducing the aggressiveness of an animal,
improving the behaviour of an animal, especially an aggressive or fearful animal,
producing a relaxing effect on the animal,
helping an animal to adapt to a new situation, for example when it is adopted, when a new animal or child arrives in its environment, when it moves house,
reducing the stress-related urine marking in an animal,
reducing the uncleanliness and the filth of an animal,
improving the conditions for familiarising an animal with its fellow creatures,
preventing the scratching or the destruction of the territory,
reducing the sound manifestations such as crying and barking,
generating a less aggressive, more relaxed and more affectionate behaviour, in particular with their masters, in an animal,

preferably for a period of more than 1 month, more advantageously more than 2 months, preferably for a period of about 3 months, the animal being a dog or a cat.

The present invention further relates to a non-therapeutic method for soothing an animal, in particular for:
ensuring the well-being of an animal in its environment,
helping an animal deal with stress and/or anxiety,
reducing the stress associated with separation in an animal,
reducing the stress associated with weaning an animal,
reducing the stress associated with loneliness in an animal,
reducing the stress in an animal before and/or during transport,
reducing the stress associated with noise such as thunderstorms or fireworks,
reducing the stress associated with a pathology, a medical treatment, a visit to a veterinarian that may require an examination or even a surgery,
reducing the aggressiveness of an animal,
improving the behaviour of an animal, especially an aggressive or fearful animal,
producing a relaxing effect on the animal,
helping an animal to adapt to a new situation, for example when it is adopted, when a new animal or child arrives in its environment, when it moves house,
reducing the stress-related urine marking in an animal,
reducing the uncleanliness and the filth of an animal,
improving the conditions for familiarising an animal with its fellow creatures,
preventing the scratching or the destruction of the territory,
reducing the sound manifestations such as crying and barking,
generating a less aggressive, more relaxed and more affectionate behaviour, in particular with their masters, in an animal,
facilitating the training and/or the education of an animal,

comprising the administration of a device according to the invention to said animal or in the immediate environment of the animal, i.e. close to its olfactory apparatus.

The present invention further relates to a semiochemical composition as defined above for preventing the manifestations of stress and/or reducing the anxiety in a pet for a period of more than 1 month, more preferably more than 2 months, preferably for a period of about 3 months, characterised in that the semiochemical composition is present in a device according to the invention.

In particular, it relates to a semiochemical composition as defined above for its use in soothing an animal, in particular for:
ensuring the well-being of an animal in its environment,
helping an animal deal with stress and/or anxiety,
reducing the stress associated with separation in an animal,
reducing the stress associated with weaning an animal,
reducing the stress associated with loneliness in an animal,
reducing the stress in an animal before and/or during transport,
reducing the stress associated with noise such as thunderstorms or fireworks,
reducing the stress associated with a pathology, a medical treatment, a visit to a veterinarian that may require an examination or even a surgery,
reducing the aggressiveness of an animal,
improving the behaviour of an animal, especially an aggressive or fearful animal,
producing a relaxing effect on the animal,
helping an animal to adapt to a new situation, for example when it is adopted, when a new animal or child arrives in its environment, when it moves house,
reducing the stress-related urine marking in an animal,
reducing the uncleanliness and the filth of an animal,
improving the conditions for familiarising an animal with its fellow creatures,
preventing the scratching or the destruction of the territory,
reducing the sound manifestations such as crying and barking,
generating a less aggressive, more relaxed and more affectionate behaviour, in particular with their masters, in an animal,
facilitating the training and/or the education of an animal,

the semiochemical composition being comprised in a device as previously defined comprising:
a polymer matrix,
a semiochemical composition,
a control agent controlling the diffusion of the semiochemical composition out of the device,
possibly an antioxidant,
possibly an anti-UV agent,
possibly a flow agent.

Preferably, the soothing semiochemical composition is administered to the animal in a device comprising:
a polymer matrix,
a semiochemical composition, in particular a soothing semiochemical composition, comprising a mixture of volatile fat acid derivatives
a control agent controlling the diffusion of the semiochemical composition out of the device.

Advantageously, the effect provided by the device is selected from:
ensuring the well-being of an animal in its environment,
reducing the stress associated with separation in an animal,
reducing the stress associated with loneliness in an animal,
reducing the stress in an animal before and/or during transport,
reducing the stress associated with noise such as thunderstorms or fireworks,
reducing the stress associated with a pathology, a medical treatment, a visit to a veterinarian that may require an examination or even a surgery,
helping an animal to adapt to a new situation, for example when it is adopted, when a new animal or child arrives in its environment, when it moves house.

Preferably and according to all the above embodiments, the pet is a cat or a dog, preferably the pet is a dog.

In a fifth aspect, the present invention relates to the use of a control agent for controlling the diffusion of a semiochemical composition into a polymer matrix to allow the release of a higher relative amount of semiochemical composition and/or for a longer period of time and/or the release of an average amount of equivalent semiochemical composition over successively at least two equal time intervals, the release of the higher amount and/or for a longer duration and/or the release of the average amount of equivalent semiochemical composition over successively at least two equal time intervals being measured relative to the amount measured when diffusing the same semiochemical composition comprised in an identical polymer matrix not containing the diffusion control agent, wherein the control agent controlling the diffusion is selected from clay, diatomite and zeolite; this results in a higher relative amount, with respect to the total amount in the device, of the semiochemical composition diffusing out of the device compared to prior art devices.

Preferably,
the control agent controlling the diffusion of the semiochemical composition out of the device is a clay, diatomite or zeolite;
the semiochemical composition is as described above, in particular composed of fat acid esters, preferably fat acid methyl esters, such as the semiochemical compositions A to U;
the polymer matrix is a thermoplastic copolymer of ethylene-vinyl acetate; and
the use of the control agent for controlling the diffusion of the semiochemical composition out of the device allows the release of a higher relative amount of semiochemical composition and/or a longer duration and/or the release of an equivalent average amount of semiochemical composition over successively at least two equal time intervals compared to the devices of the prior art, in particular, the release of a higher amount of semiochemical composition and/or for a longer duration and/or the release of an equivalent average amount of semiochemical composition over successively at least two equal time intervals lasts for more than 1 month, preferably for a duration of about 3 months:
the pet is a dog.

Advantageously, the control agent controlling the diffusion of the semiochemical composition out of the device is chosen from clays with a chain structure (chains of silica tetrahedra similar to hornblende), in particular phyllosilicates such as wollastonite (for example sold under the name casiflux F75), bentonite, kaolinite, montmorillonite, attapulgite, sepiolite and paligorskyte. Preferably attapulgite, sepiolite and paligorskyte, even more preferably the agent is sepiolite.

The devices according to the invention, whatever their form, are prepared by mixing the ingredients of which they are composed, and this mixture can then be shaped by various means known to the person skilled in the art: in particular, by extrusion, moulding or even 3D printing.

The manufacture by moulding, possibly by injection moulding, requires the mixture of ingredients making up the device to be melted and then introduced into a mould having the shape of the finished product, this mould may be coated with a product which facilitates demoulding, the molten mixture cools and solidifies, the finished product is then unmolded. The unmolded finished product may be rectified by cutting or sanding to remove the imperfections.

The 3D printing is preferably implemented by “Fused Deposition Modeling” (FDM); this technique consists in depositing layer by layer a filament of the molten mixture of ingredients which, by superimposing, gives shape to the object. The print head moves according to the coordinates X, Y and Z (length, width and height) transmitted by a 3D file corresponding to the 3D model of the object to be printed.

The principle of operation of the extrusion manufacturing method consist in pushing the mixture of ingredients through a die. The shape of the die corresponds to the cross section of the finished part.

The implementation of this extrusion method thus comprises:
the mixture of ingredients in the device;
the extrusion of this mixture in an extruder leading to an extruded product;
optionally, the calendering of the extruded product;
the cutting allowing to obtain the finished product.

According to an illustrative embodiment, the mixing step of the ingredients consists in introducing the polymer (preferably 9% EVA) into a ploughshare mixer, for example, and then adding the semiochemical composition under stirring. The addition can for example be done by spraying, with a jet of the semiochemical composition. The control agent controlling the diffusion of the semiochemical composition out of the device is then added (preferably sepiolite). Everything is mixed together. Then, if present, the flow agent is added to the mixture (preferably 1% colloidal silica) and the whole is stirred and sieved. Subsequently, a further dose of the flow agent is added to the sieve for sieved (preferably 0.4% colloidal silica). Finally, the second half of the polymer (preferably 9% EVA) is added to the reversal mixer and everything is mixed together again.

The mixture of ingredients is then fed into an extruder which can be single or twin screw. If necessary, the extruded web is calendered so as to obtain the exact dimensions required; the calendering also allows the extruded web to cool down so as to obtain a solid product.

The extruded product is in the form of a ribbon which is then cut in order to obtain the finished product.

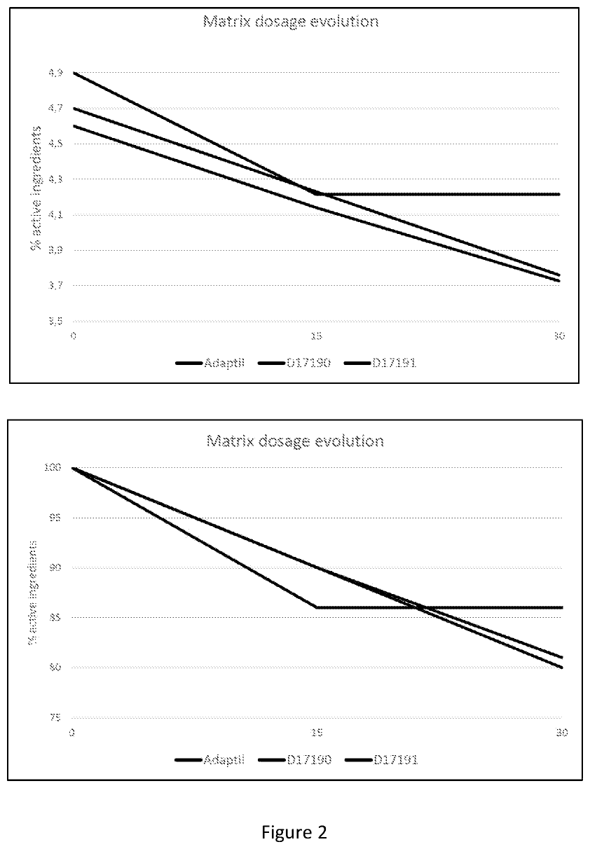

### FIGURES

[FIG. 1]: volatile fat acid ester derivative

[FIG. 2]: release of fat acid methyl esters over time (example 2A).

### EXAMPLES

### Example 1

### Preparation of Soothing Dog Collars According to the Invention

### Example 1A

The collar device of the present invention consists of a mixture comprising semiochemical actives (mixture R of fat acid methyl esters comprising methyl laurate, methyl myristate, methyl palmitate, methyl stearate, methyl oleate and methyl linoleate), an ethylene-vinyl acetate (EVA) polymer matrix (9% EVA), a clay-type control agent controlling the diffusion of the semiochemical composition out of the device (18.5% montmorillonite) and a flow agent (1% colloidal silica and then 1% sequentially).

### Preparation:

The first step consists of introducing half of the polymer (9% EVA) into a ploughshare mixer and then add the mixture of fat acid methyl esters by spraying under stirring. Montmorillonite is then added. Everything is mixed for 30 seconds.

A second step consists in adding the flow agent to the mixture (1% colloidal silica), mixing for 30 seconds and then sieving the mixture. Subsequently, another dose of the flow agent is sieved into the mixture (1% colloidal silica).

Finally, the second half of the polymer (9% EVA) is added to the reversal mixer and the whole is mixed for another 3 minutes.

This mixture is then shaped by extrusion.

### Example 1B

The collar device of the present invention consists of a mixture comprising semiochemical actives (mixture R of fat acid methyl esters comprising methyl laurate, methyl myristate, methyl palmitate, methyl stearate, methyl oleate and methyl linoleate), an ethylene-vinyl acetate polymer matrix (9% EVA), a clay-type control agent controlling the diffusion of the semiochemical composition out of the device (15% sepiolite) and a flow agent (01.9% colloidal silica).

### Preparation:

In a first step, the sepiolite and the colloidal silica are mixed in a ploughshare mixer. Then the mixture of fat acid methyl esters is added under stirring. The whole thing is mixed for 20 seconds and sieved. The mixture is transferred to an reversal mixer containing 9% EVA and the whole is mixed for 3 minutes.

This mixture is then shaped by extrusion.

### Examples 1C (batch D17190), 1D (batch D17191), 1E (batch D18001) and 1F (batch

D17207)

### Collars 1C, 1D, 1E:

The collar device of the present invention consists of a mixture comprising semiochemical actives (methyl laurate, methyl myristate, methyl palmitate, methyl stearate, methyl oleate and methyl linoleate), an ethylene-vinyl acetate type polymer matrix (9% EVA for example 1C and 1E, EVA at 16% for example 1D), a control agent controlling the diffusion of the semiochemical composition out of the device of the clay type (15% sepiolite), a flow agent (colloidal silica at 1%, then at 0.4% sequentially), an antioxidant (0.005% TBHQ), and an anti-UV agent for example 1E only (1% Hostavin N30 P).

### Collar 1F:

The collar device of the present invention consists of a mixture comprising semiochemical actives (mixture R of fat acid methyl esters comprising methyl laurate, methyl myristate, methyl palmitate, methyl stearate, methyl oleate and methyl linoleate), an ethylene-vinyl acetate type polymer matrix (9% EVA), a clay-like control agent controlling the diffusion of the semiochemical composition out of the device (15% sepiolite), a flow agent (1.4% colloidal silica), and an antioxidant (0.005% TBHQ).

### Preparation:

Examples 1C, 1D, 1E and 1F were prepared according to the same preparation mode.

Half of the EVA was introduced into the ploughshare mixer. In addition, the TBHQ antioxidant was dissolved in the mixture of fat acid methyl esters, and this mixture was sprayed under stirring onto the EVA in a jet perpendicular to the axis. The sepiolite as a control agent controlling the diffusion of the semiochemical composition out of the device was then added and the whole was mixed for 30 seconds. Then, 1% colloidal silica (+anti-UV agent (Hostavin N30 P) for example E) was introduced while mixing for another 30 seconds. The mixture was then sieved through a 1 mm mesh sieve.

A further dose of colloidal silica (0.4%) was then sieved into the mixture. This was transferred to the second half of the EVA, in a reversal mixer. The whole thing was finally mixed for 3 minutes.

This mixture is then shaped by extrusion.

### Summary of Examples 1A to 1F

TABLE 1

| | | | | control agent | | | |
|---|---|---|---|---|---|---|---|
| | | | | controlling the | | | |
| | | | | diffusion of the | | | |
| | | | | semiochemical | | | |
| | | Semiochemical | Polymer | composition out | Flow | Anti-UV | |
| Example | Batch | composition* | matrix | of the device | agent | agent | Antioxidant |
| 1A | D17061 | Mixture R | EVA | montmorillonite | colloidal | no | no |
| | | (5.21%) | (VA 9%) | (18.5%) | silica | | |
| | | | | | (2%) | | |
| 1B | D17066 | Mixture R | EVA | sepiolite (15%) | colloidal | no | no |
| | | (4.82%) | (VA 9%) | | silica | | |
| | | | | | (1.9%) | | |
| 1C | D17190 | Mixture R | EVA | sepiolite (15%) | colloidal | no | yes (TBHQ |
| | | (4.87%) | (VA 9%) | | silica | | 0.005%) |
| | | | | | (1.4%) | | |
| 1D | D17191 | Mixture R | EVA | sepiolite (15%) | colloidal | no | yes (TBHQ |
| | | (4.87%) | (VA 16%) | | silica | | 0.005%) |
| | | | | | (1.4%) | | |
| 1E | D18001 | Mixture R | EVA | sepiolite (15%) | colloidal | yes | yes (TBHQ |
| | | (4.87%) | (VA 9%) | | silica | (Hostavin | 0.005%) |
| | | | | | (1.4%) | N30 P 1%) | |
| 1F | D17207 | Mixture R | EVA | sepiolite (15%) | colloidal | no | yes (TBHQ |
| | | (4.87%) | (VA 9%) | | silica | | 0.005%) |
| | | | | | (1.4%) | | |

*methyl laurate, methyl myristate, methyl palmitate, methyl stearate, methyl oleate, methyl linoleate

### Example 2

### Characterisation of the Release Profile of the Semiochemical Composition (FAME)

### Example 2A—Study of the In Vivo Release for 1 Month

A comparative study of different collars containing a semiochemical composition was conducted in dogs in order to measure the release kinetics of the composition and to verify the good local tolerance of the collars in the animal.

For this purpose, three collars of different compositions were tested: the commercial control collar ADAPTIL® batch P098663/099295, the collar prepared according to example 1C (batch D17190) and a collar prepared according to example 1D (batch D17191).

Thus, three groups of eight dogs each wore one of the three collars for fourteen days or twenty-eight days (4 dogs per timeline per group).

At the end of each of the timelines, the fat acid methyl esters still present in the collars were dosed as described in Example 2A.

The results of the release of the fat acid methyl esters over time are presented in the following table and in FIG. 2:

TABLE 2

| | T0 | T14d | T28d |
|---|---|---|---|
| Control collar | 100% | 86% | 86% |
| Collar 1C (EVA 9%) | 100% | 90% | 81% |
| Collar 1D (EVA 16%) | 100% | 90% | 80% |

After 14 days, the amount of fat acid methyl esters released was not significantly different between the control collar and the two collars tested according to the invention.

On the other hand, after 28 days, the two collars tested according to the invention contain less fat acid methyl esters, indicating that the collars according to the invention make more fat acid methyl esters available than at 14 days, in contrast to the control collar whose amount of fat acid methyl esters released does not vary significantly.

Between T0 and T28d, the quantity of methyl esters in the collar according to the invention decreases regularly, by 10% per 14-day period, which is not the case in the control collar.

### Example 2B—Study of the In Vivo Release for 3 Months

A comparative in vivo study was carried out, over a period of three months, with the objective of evaluating the time during which a device according to the invention is capable of releasing the soothing semiochemical composition.

This time the commercial control collar ADAPTIL® batch P100736/101294 was compared to batch D18001 (sepiolite+EVA 9%) prepared according to example 1E.

Thus, a total of sixteen dogs were divided into two groups and each dog wore one of the two collars for 1 or 3 months (4 dogs per timeline and per group).

At the end of each timeline, the actives were dosed in matrix and the results are presented in the following table:

TABLE 3

| | Collar 1E | Control collar |
|---|---|---|
| T1M | 79% | 86% |
| T3M | 52% | 72% |

The results of this study showed that:
The collar according to the invention allows a release for at least three months
The collar according to the invention allows a greater availability of the semiochemical composition than the control for at least three months.

### Example 3

### Study of the Stability of the Composition for 3 Months

The stability of a first series of formulas was tested over three months to compare the influence of the control agent controlling the diffusion of the semiochemical composition out of the device (sepiolite or montmorillonite) and of the vinyl acetate (VA) level in the EVA copolymer (9% or 16%). The formulas were stored unpackaged in the climate chambers.

TABLE 4

| | | | control agent controlling |
|---|---|---|---|
| | | | the diffusion of the |
| | | | semiochemical |
| | Semiochemical | Polymer | composition out of the |
| Batch | composition* | matrix | device |
| D17126 | 5% mixture R | EVA (VA 9%) | montmorillonite (18.5%) |
| D17127 | 5% mixture R | EVA (VA 9%) | sepiolite (15%) |
| D17129 | 5% mixture R | EVA (VA 16%) | sepiolite (15%) |

TABLE 5

| | 25° C./60% RH | | 40° C./75% RH | |
|---|---|---|---|---|
| | T1 month | T3 months | T15 days | T1 month |
| D17126 | 102.5% | 94.9% | 93.4% | 92.6% |
| (montmorillonite | T0 | T0 | T0 | T0 |
| base/VA 9%) | | | | |
| D17127 (sepiolite | 94.6% | 90.3% | 93.8% | 99.0% |
| base/VA 9%) | T0 | T0 | T0 | T0 |
| D17129 (sepiolite | 106.1% | 100.3% | 106.1% | 108.2% |
| base/VA 16%) | T0 | T0 | T0 | T0 |

The sepiolite-based batches show homogeneous results throughout the stability study, whether they are formulas with EVA with VA 9% or 16%. The montmorillonite and EVA with 9% VA based batch shows an acceptable stability after 3 months.

For the first series, this stability showed that over three months, the sepiolite-based batches are more stable in terms of semiochemical composition content.

Then, the stability of a second series of formulas based on sepiolite, 9% EVA and several additives (antioxidant, anti-UV, dispersant) was tested. In particular, the additives were introduced separately to study their effect on the stability. The effect of a higher concentration of active ingredient was also studied (5% or 10%). The formulas were stored unpackaged in the climate chambers. In addition, the formulas stored at 40° C./75% RH were exposed to UV light for 8 hours a day during 1 month of storage.

TABLE 6

| | | | control agent | | | |
|---|---|---|---|---|---|---|
| | | | controlling the | | | |
| | | | diffusion of the | | | |
| | | | semiochemical | | | |
| | Semiochemical | Polymer | composition out | Flow | Anti-UV | |
| Batch | composition* | matrix | of the device | agent | agent | Antioxidant |
| D17161 | 10% mixture R | EVA | Sepiolite | Colloidal | — | — |
| | | (VA 9%) | (30%) | silica | | |
| D17162 | 5% mixture R | | Sepiolite | (1.4%) | Hostavin ARO | — |
| | | | (15%) | | 8P (0.5%) | |
| D17163 | 5% mixture R | | | | Hostavin | — |
| | | | | | N30P (0.5%) | |
| D17164 | 5% mixture R | | | | — | TBHQ |
| | | | | | | (0.005%) |

TABLE 7

| | 25° C./60% RH | | 40° C./75% RH | |
|---|---|---|---|---|
| | T1 month | T3 months | T15 days | T1 month |
| D17161 (FAME | — | 104.8% T0 | 100.5% T0 | 103.7% T0 |
| 10%) | | | | |
| D17162 (0.5% | 91.5% T0 | 94.6% T0 | 93.0% T0 | 93.0% T0 |
| Anti UV 1) | | | | |
| D17163 (0.5% | — | 101.8% T0 | 98.9% T0 | 102.4% T0 |
| Anti UV 2) | | | | |
| D17164 (0.005% | — | 98.9% T0 | 98.1% T0 | 92.6% T0 |
| Anti-oxidant) | | | | |

For the second series, it was shown that collars exposed for 8 hours a day to UV light were stable. The presence of an anti-UV agent or an antioxidant is therefore optional.

### Example 4

### In Vivo Study: Perception of Effectiveness

A consumer study was carried out in the homes of dog owners in France, in particular by evaluating the effectiveness of a collar according to the present invention as a complement or not to a pest control collar.

Dog owners were then questioned via questionnaires about their perception of different behavioural criteria of their dog.

These dogs were one in number in their household, not cohabiting with a cat, over three months of age, females and males together, the majority of them not neutered/sterilised, none pregnant or lactating, no outdoor exercise restrictions, no skin problems or skin allergies, not being treated for behavioural problems, but with minor behavioural problems.

The home test of the collars lasted three months and the households were divided into two groups of 75 dogs in total: one group of 45 dogs received a batch D18101 collar (wearing the same collar for three months), and the second group of 30 dogs received a batch D18101 collar and a pest control collar (wearing both collars simultaneously for three months). In each of these groups, 50% of the dogs weighed less than 10 kg and 50% of the dogs weighed more than 10 kg.

At the end of the three months, there was a positive impact of the collars on signs of stress or anxiety throughout the collar wearing period, while no significant difference in behaviour was found between the two groups of dogs. It should also be noted that wearing the collar of batch D18101 according to the invention does not alter the effectiveness of the pest control collar and vice versa.

1. A device for continuously releasing onto an animal or into the immediate environment of an animal a semiochemical composition for pets comprising:
a polymer matrix,
a semiochemical composition comprising a mixture of volatile fat acid derivatives,
a control agent controlling the diffusion of the semiochemical composition out of the device selected from a clay, a diatomite and a zeolite.

2. The device according to claim 1, the device being a collar.

3. The device according to claim 1, wherein the control agent controlling the diffusion is selected from attapulgite, sepiolite and paligorskyte.

4. The device according to claim 1, wherein the control agent controlling the diffusion of the semiochemical composition out of the device is present in the device in proportions varying from 5 to 25%, advantageously from 10 to 20%, preferably about 15% by weight with respect to the total weight of the device.

5. The device according to claim 4, wherein the polymer matrix is a thermoplastic copolymer of ethylene-vinyl acetate.

6. The device according to claim 1, wherein the semiochemical composition comprises a mixture of volatile fat acid derivatives selected from the group consisting of methyl oleate, methyl palmitate, methyl azelate, methyl pimelate, methyl caprate, methyl laurate, methyl myristate, methyl palmitoleate, methyl linoleate, methyl stearate, methyl arachidonate, methyl n-butyrate, methyl isobutyrate, methyl α-methylbutyrate, methyl caproate, methyl pivalate, methyl γ-linoleate, methyl eicosapentanoate, methyl pentadecanoate, methyl tridecanoate, methyl docosahexanoate, advantageously selected from the group consisting of methyl oleate, methyl palmitate, methyl laurate, methyl myristate, methyl linoleate, methyl stearate and methyl pentadecanoate.

7. The device according to claim 1, wherein the semiochemical composition is a mixture of fat acid esters comprising between 32% and 38% methyl oleate, between 1% and 3% methyl laurate, between 13% and 16% methyl stearate, between 18% and 24% methyl linoleate, between 3% and 7% methyl myristate, and between 18% and 24% methyl palmitate, by weight based on the total weight of the semiochemical composition.

8. The device according to claim 1, wherein the semiochemical composition is between 0.1% and 25% by weight of the total weight of the device, advantageously from 1 to 10% by weight, more advantageously from 3 to 8% by weight, preferably 5% by weight of the total weight of the device.

9. The device according to claim 1, further comprising a flow agent such as colloidal silica and an ingredient allowing to improve the stability over time of the semiochemical composition such as an antioxidant or an anti-UV agent.

10. A semiochemical composition present in a device according to claim 1, for its use in preventing the manifestations of stress and/or reducing the anxiety in a pet for a period of more than 1 month, more preferably more than 2 months, preferably for a period of about 3 months.

11. A semiochemical composition present in a device according to claim 1 for its use in preventing the manifestations of stress and/or reducing the anxiety in a pet for a period of more than 1 month, more preferably more than 2 months, preferably for a period of about 3 months, the pet being a dog.

12. A use of a control agent for controlling the diffusion of a semiochemical composition in a device according to claim 1 to allow for the regular diffusion over time and/or the diffusion of a higher relative amount of said semiochemical composition comprising a mixture of volatile fat acid derivatives out of said polymer matrix, the regular diffusion and/or the diffusion of a higher relative amount being measured in relation to the amount measured when diffusing the same semiochemical composition comprised in an identical polymer matrix not containing the control agent controlling the diffusion, characterised in that the control agent controlling the diffusion is selected from clay, diatomite and zeolite.

13. The use according to claim 12, wherein the control agent controlling the diffusion is a phyllosilicate selected from attapulgite, sepiolite and paligorskyte.

14. The use according to claim 12, wherein the polymer matrix is a thermoplastic copolymer of ethylene-vinyl acetate.
